(19) United States
(12) Patent Application Publication
(10) Pub. No.: US 2021/0332000 A1
(43) Pub. Date: Oct. 28, 2021

(54) Ethyl Benzyl Quaternary Amines of Amido Amines for Improved Antifungal properties

(71) Applicant: Thomas P. Daly, Arlington Heights, IL (US)

(72) Inventor: Thomas P. Daly, Arlington Heights, IL (US)

(21) Appl. No.: 17/372,431

(22) Filed: Jul. 10, 2021

Related U.S. Application Data
(63) Continuation-in-part of application No. 17/370,992, filed on Jul. 8, 2021.
Application No. 17/370,992 is a continuation of application No. 16/430,497, filed on Jun. 4, 2019.
Application No. 16/430,497 is a continuation of application No. 15/725,757, filed on Oct. 5, 2017, now Pat. No. 10,308,589.
Application No. 15/725,757 is a continuation-in-part of application No. 14/969,920, filed on Dec. 15, 2015, now Pat. No. 9,802,908.
(60) Provisional application No. 63/050,683, filed on Jul. 10, 2020.
(60) Provisional application No. 62/094,741, filed on Dec. 19, 2014.

(51) Int. Cl. C07C 211/63; C07C 233/36

(52) U.S. Cl. CPC C07C 211/63; C07C 233/36

(57) ABSTRACT

Ethylbenzyl chloride quats (EB quats) and their related salts. The synthesis of EB quats requires minimal capital requirements, produces products in good yields, without difficult to dispose of waste and in a cost-effective manner. The preferred embodiment of the invention is to perform a condensation reaction of a linear or branched, saturated or unsaturated fatty acid, of between 2 and 22 carbons with an amine that has either a primary amine and tertiary amine or a secondary amine and tertiary amine. An example is dimethylaminopropylamine (DMAPA). The amido amine that results from the condensation reaction described is then reacted with ethyl benzyl chloride (EBC) to produce the desired EB quat. Additional processing, such as ion exchange can be performed to eliminate the chlorine or substitute it for another anionic species, organic or inorganic.

This application hereby incorporates by reference application Ser. No. 17/370,992 filed Jul. 8, 2021 and 63/050,683 filed Jul. 10, 2020 in their entireties.

### BACKGROUND

### Field of the Invention

The present invention relates to the field of amines, quaternary amines and more particularly to ethylbenzyl (EB) quaternary amines.

### Description of the Problem Solved by the Invention

Quaternary amines are very useful compounds as cationic surfactants, corrosion inhibitors, personal care emollients, electrolytes and antimicrobials. Typical methyl and benzyl quaternary amines are typically much more antibacterial than antifungal, and have little effect against spores. Ethyl Benzyl quaternaries have much greater fungal and spore efficacy, as well as effectiveness against viruses, and mycobacteria. The production of many such ethyl benzyl quats require a large investment in high pressure equipment to produce the tertiary amines needed as precursors. The present invention utilizes fatty acids and amines with both a primary and tertiary amine group, or a secondary and a tertiary amine group. The readily available nature and cost effectiveness of these amines makes these products not only more effective, but also cost effective to the smaller chemical manufacturer.

### SUMMARY OF THE INVENTION

The present invention relates to ethylbenzyl chloride quats and their related salts. The synthesis of EB quats requires minimal capital requirements, produces products in good yields, without difficult to dispose of waste and in a cost-effective manner. The preferred embodiment of the invention is to perform a condensation reaction of a linear or branched, saturated or unsaturated fatty acid, of between 2 and 22 carbons with an amine that has either a primary amine and tertiary amine or a secondary amine and tertiary amine. The example used herein will be dimethylaminopropylamine (DMAPA), but the invention is not limited to solely DMAPA but to any amine as described. The amido amine that results from the condensation reaction described is then reacted with ethyl benzyl chloride (EBC) to produce the desired EB quaternary. Additional processing, such as ion exchange can be performed to eliminate the chlorine or substitute it for another anionic species, organic or inorganic. Acetates are the simplest organic salt of the EB quaternaries. Propionates and butyrates result in even greater antifungal properties than the chloride salts.

The reaction of ethylbenzyl chloride with amidoamines produces EB quaternaries that are superior to their methyl and benzyl quat analogs in that they are more antifungal and antiviral. The invention disclosed herein is a cost-effective way to may quaternaries that are mild to the skin, non-hazardous and have multiple uses. Applications for such quaternaries are in personal care as a treatment for acne, dandruff, psoriasis, and other skin born bacterial or fungal maladies. Additionally, the EB quats are much improved fungicides and sporicides for use in agriculture. In oilfield applications, the EB quaternaries are useful as surface treatments for clays, such as bentonite, and as corrosion inhibitors and power improvers.

### DESCRIPTION OF THE FIGURES

Attention is now directed to several drawings the illustrate features of the present invention.

FIG. 1 shows the synthesis of EB quaternary from amidoamines, n is an integer. R is alkyl, linear or branched, saturated or unsaturated, cyclic or acylic from 1 to 21 carbons.

FIG. 2 shows the use of dimethylpropylamine as the amidoamine. R is alkyl, linear or branched, saturated or unsaturated, cyclic or acylic from 1 to 21 carbons.

FIG. 3 shows the amidoamine quaternary from lauric acid and DMAPA. R is alkyl, linear or branched, saturated or unsaturated, cyclic or acylic from 1 to 21 carbons.

FIG. 4 shows the use of aminoethylpiperadine as the diamine. R and R¹ are independently chosen from alkyl, linear or branched, saturated or unsaturated, cyclic or acylic from 1 to 21 carbons.

FIG. 5 shows the synthesis of dialkyl amidoamine quaternaries. R and R¹ are independently chosen from alkyl, linear or branched, saturated, cyclic or acylic or unsaturated from 1 to 21 carbons.

FIG. 6 shows the synthesis of dialkyl quats from N,N-Bis(2-aminoethyl)methylamine. R and R¹ are independently chosen from alkyl, linear or branched, saturated or unsaturated, cyclic or acylic from 1 to 21 carbons.

FIG. 7 shows the synthesis of dialkyl quats from N-(3-aminopropyl)-N-methylpropane-1,3-diamine. R and R¹ are independently chosen from alkyl, linear or branched, saturated or unsaturated, cyclic or acylic from 1 to 21 carbons.

FIG. 8 shows the synthesis of amidopoyamines and their zwitterionic derivatives.

FIG. 9 shows the synthesis of a wider range of amido tertiary amines where the tertiary amine is not a dimethyl amine. R, R¹ and R² are independently chosen from alkyl, linear or branched, saturated or unsaturated, cyclic or acylic from 1 to 21 carbons.

FIG. 10 shows the synthesis of amidoamine quats from the quatting agents ethylene oxide, chloropropyltrishydroxysilane and acrylates. R, R¹ and R² are independently chosen from alkyl, linear or branched, saturated or unsaturated, cyclic or acylic from 1 to 21 carbons, n and m are integers greater than zero, and G is chosen from —H, —CH₃, —CH₂CH₃, or —OH.

Several figures and illustrations have been provided to aid in understanding the present invention. The scope of the present invention is not limited to what is shown in the figures.

### DESCRIPTION OF THE PREFERRED EMBODIMENTS

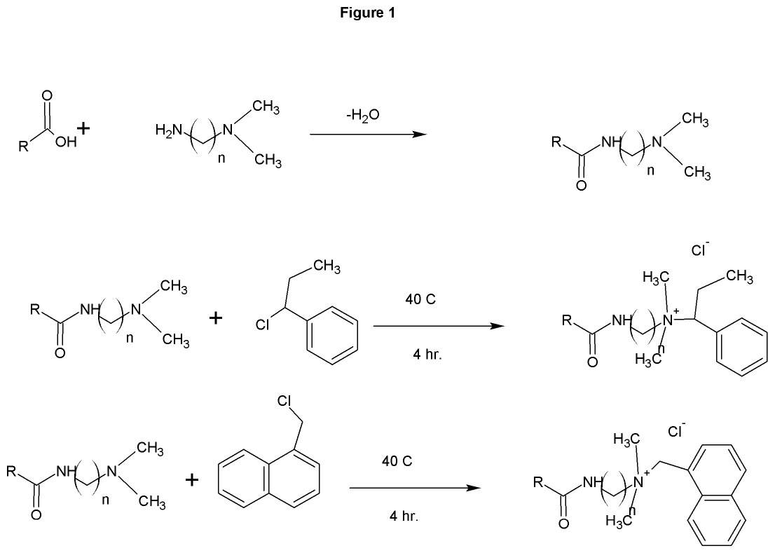

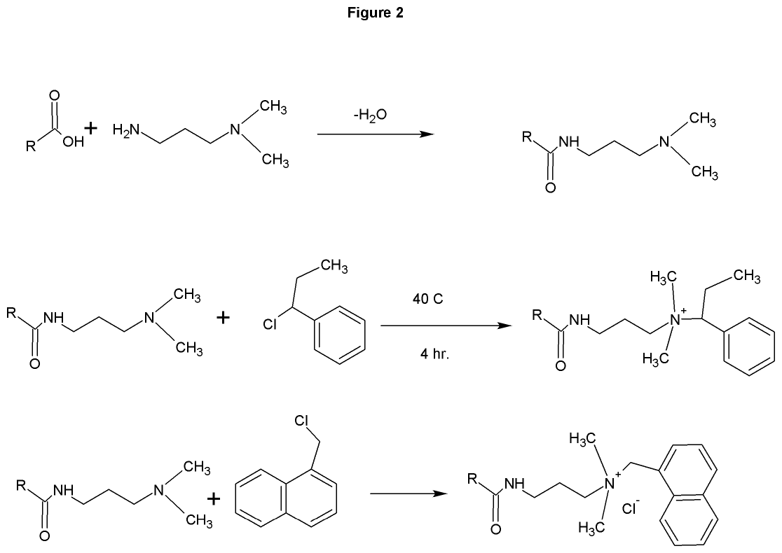

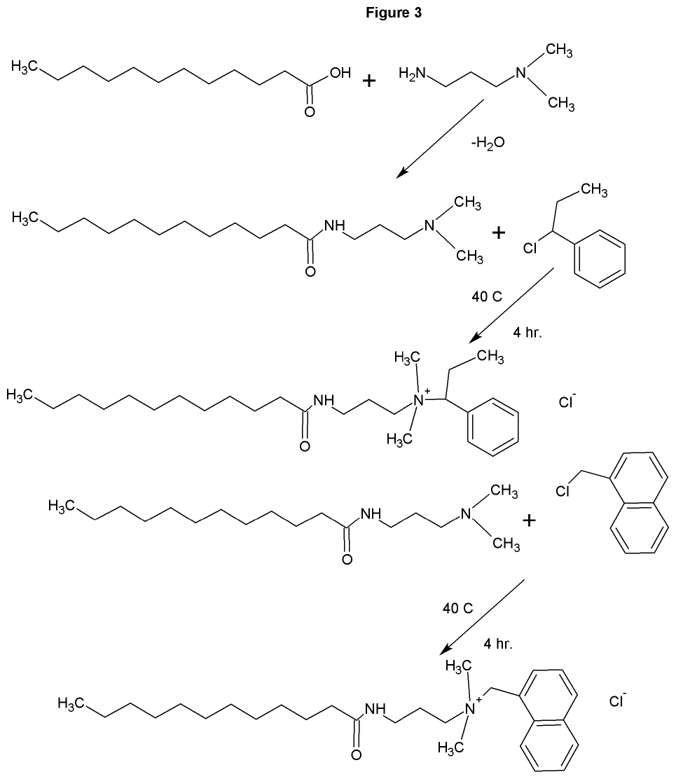

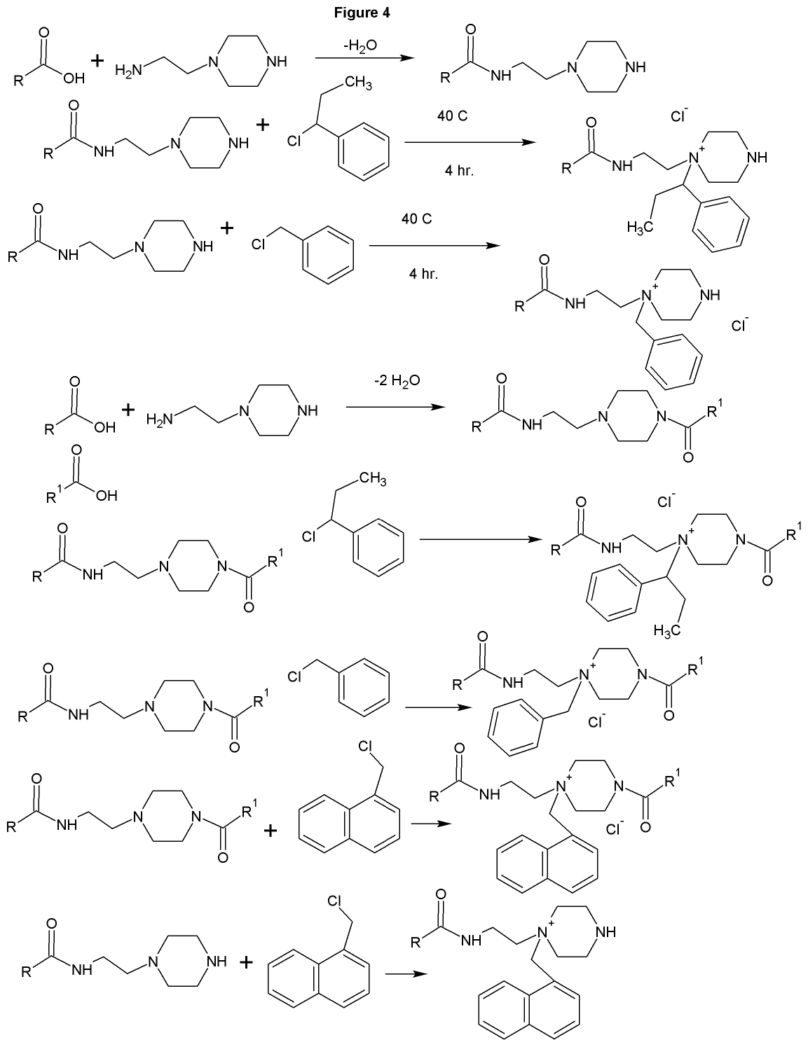

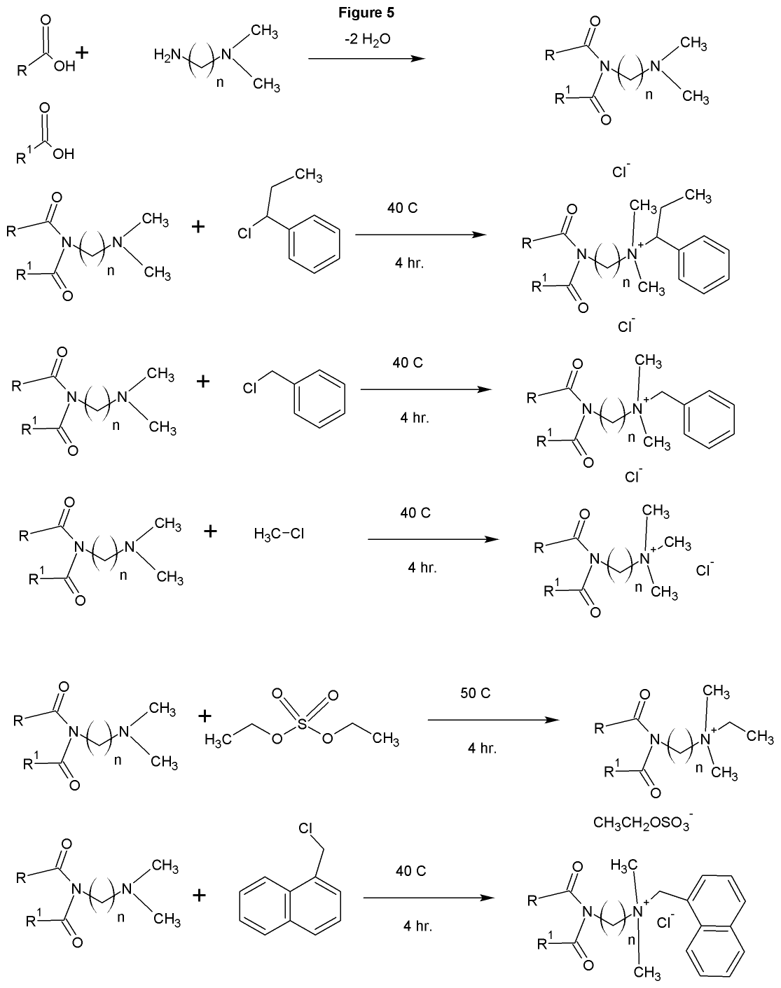

Ethyl benzyl quaternaries (EB quats) and their salts, as well as salt free EB quats offer a distinct advantage over methyl and benzyl quaternaries. The EB quats have superior anti-fungal performance and, the amidoamine quats, can be made in cost effective manner with minimal capital. FIG. 1 shows the general principle, showing a linear, alkyl diamine, but this need not be the case. However, the preferred embodiment of the invention is the condensation of fatty acid and dimethylaminopropyl amins (DMAPA), followed by reaction with ethyl benzyl chloride as shown in FIGS. 2 and 3. Other diamines and even other polyamines so long as a primary or secondary amine is present and a tertiary amine. Another example would be the use aminoethylpiperadine as taught in FIG. 4. FIG. 5 shows the synthesis of dialkyl amidoamine quaternaries. The diamido compounds are stearically hindered and thus, more difficult to prepare. A longer reaction time is required to reach the diamide as the temperature must be mostly held at around 80 C under about 20 LB of vacuum. Higher temperatures in the DMAPA case, will lead to loss of DMAPA. Activated forms of fatty acids may also be utilized, such as acid chlorides (sufficient base neutralization must occur during the reaction or sufficient base must be present before the reaction to neutralize the HCl generated and prevent amine neutralization. Aldehydes may also be used. Starting from oils did result in some diamide, but not in sufficient quality versus the longer the reaction time. The ethyl quaternary is formed with an ethyl sulfate salt for the purposes of demonstrating that various counter anions are possible and all are within the scope of the invention.

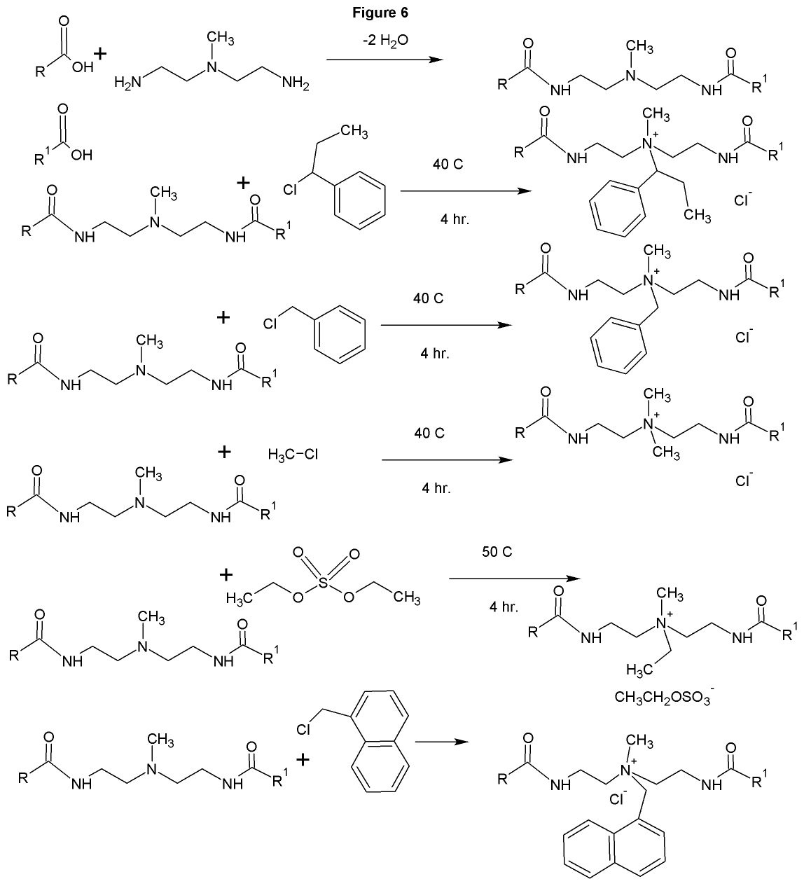

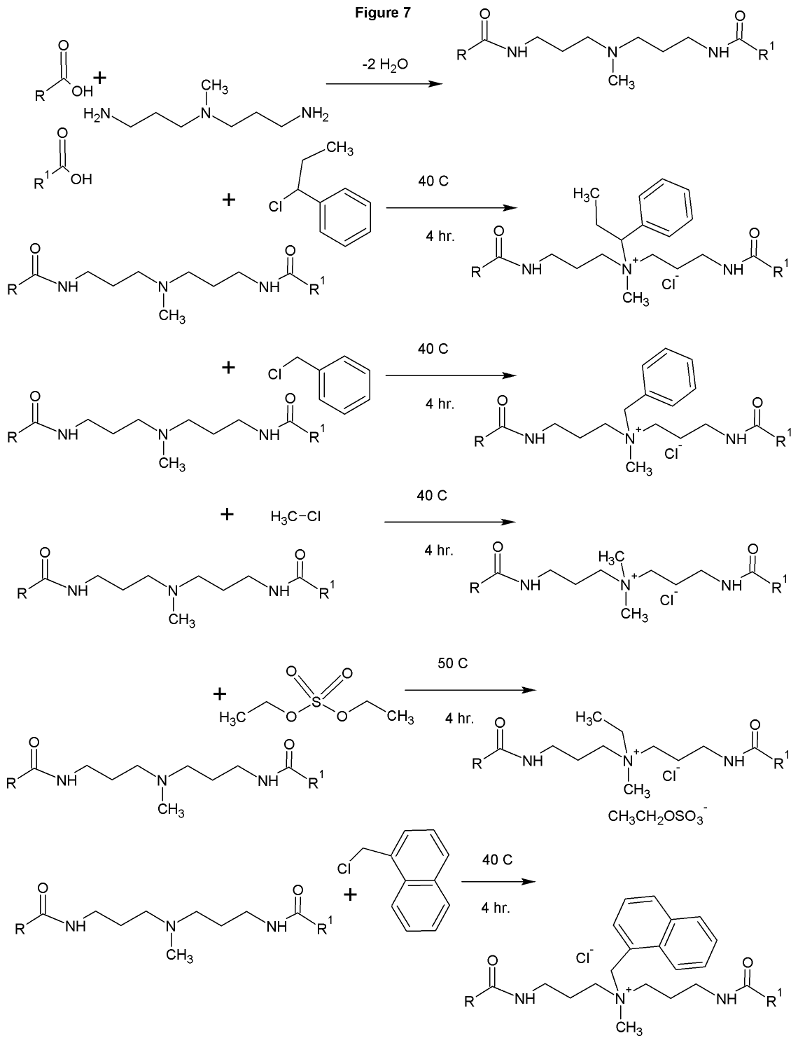

Another way of achieving dialkyl amidoamine quats is to condense N,N-Bis(2-aminoethyl)methylamine with two moles of fatty acid, followed by quaternization of the tertiary amine as shown in FIG. 6. FIG. 7 shows the synthesis of dialkyl quats from N-(3-aminopropyl)-N-methylpropane-1,3-diamine, which has the advantage of being easier to prepare than N,N-Bis(2-aminoethyl)methylamine. N-(3-aminopropyl)-N-methylpropane-1,3-diamine is readily made in good yield by reacting 2 moles of acrylonitrile with methyl amine and reducing with hydrogen over a sponge nickel catalyst.

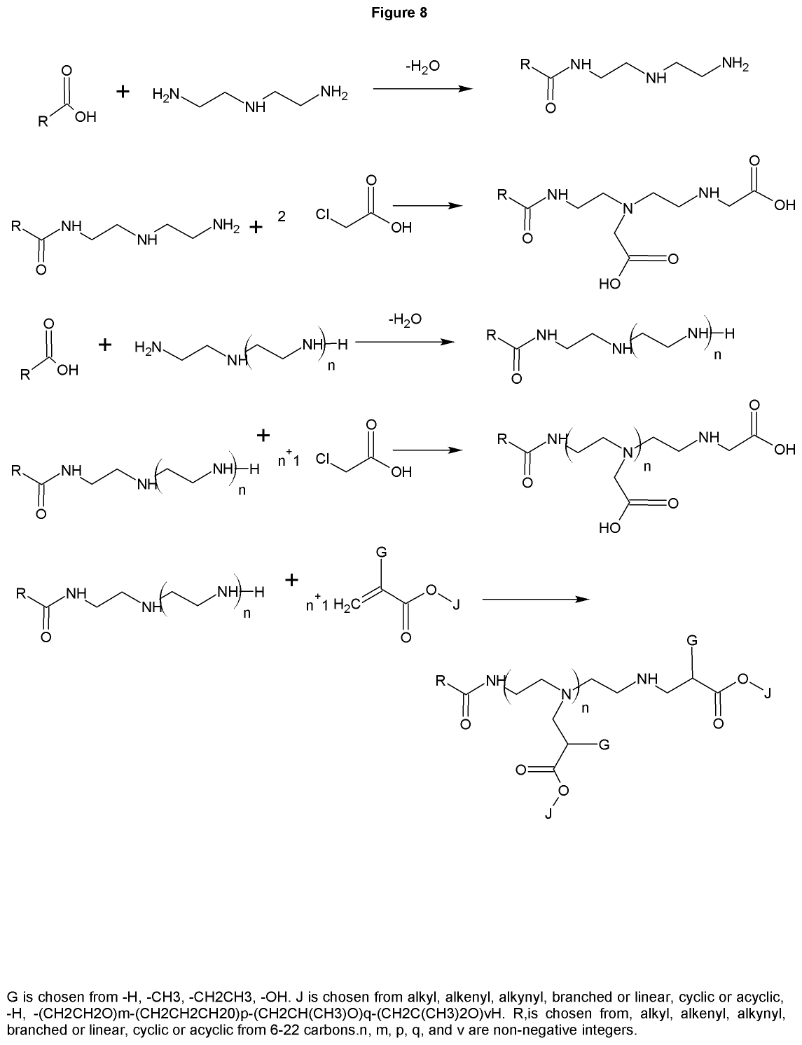

FIG. 8 shows the synthesis of amidopolyamines and their zwitterionic derivatives. The polyzwitterionic amines are useful as asphalt emulsifiers and conditioners when the asphalt is difficult to emulsify. The molecules of FIG. 8 are also useful surfactants in harsh pH environments and are suitable for use and micelle formation in both acidic and basic environments.

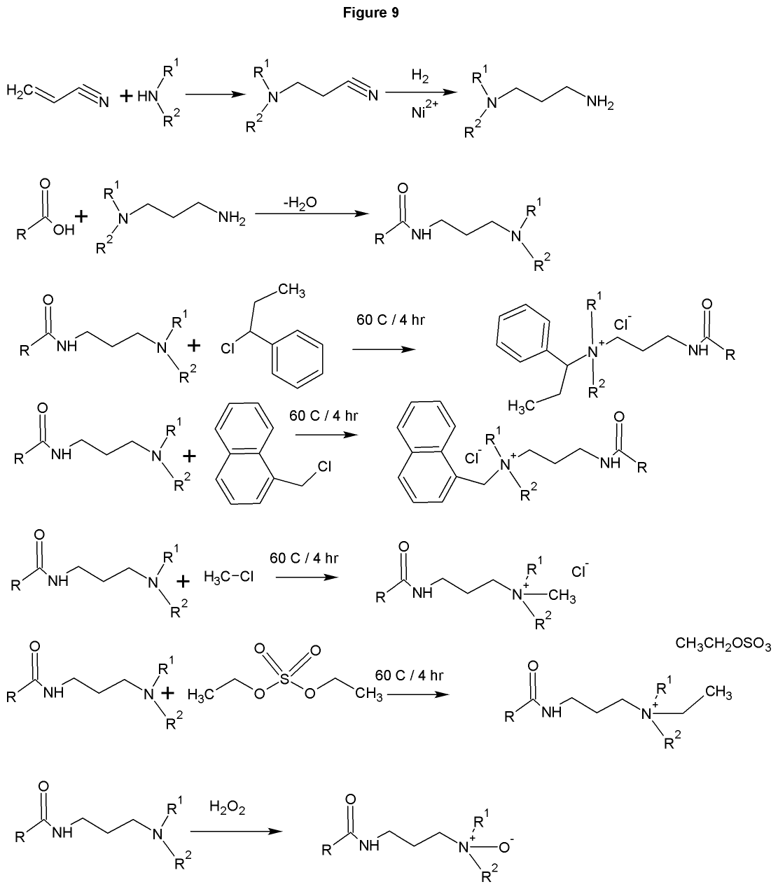

FIG. 9 shows the synthesis of tertiary amidoamines where the tertiary amine is not a dimethyl. Any secondary amine can be used to react with acrylonitrile to form the difunctional primary/tertiary amine to be quaternarized. In cases where R¹ and R² are in the range of greater than 6, the amidoamines are well suited to be pour point depressants and the quats are suited to “gunker” type corrosion inhibitors.

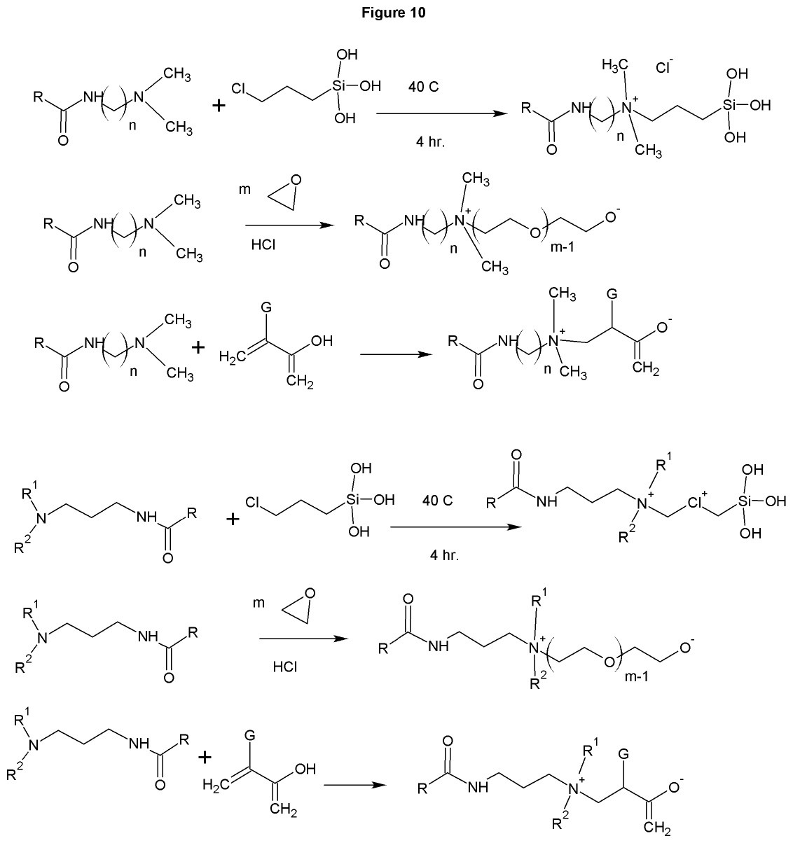

FIG. 10 shows the synthesis of amidoamine quats utilizing chloropropyltrishydroxysilane, ethylene oxide and acrylates. Alkoxylates of acrylic acid as well as hydroxyacrylic, methacrylic and ethacrylic acid may also be utilized as they also form a zwitterion and the reactions proceed favorably. While still effective sanitizing agents, the chloropropyltrishydroxysilane and the alkoxylated acrylates, especially the propoxylated and butoxylated acrylates, form a film on surfaces that corrosion causing bacteria have difficulty binding to and forming colonies, giving them improved corrosion inhibition over traditional corrosion inhibitors.

The molecules described in this invention disclosure are useful in a range of applications. The molecules find utility as fungicides in personal care as treatments for acne, dandruff, psoriasis, and other fungal skin born conditions, as well as use in feminine products where an antifungal is required that is gentle on the sensitive tissues, as well as HIV prevention. The molecules described herein are also excellent hair conditioners and laundry fabric softeners. Other applications include agriculture as a sporicide, algicide, and fungicide. Oil field applications include treatment of clay to make hydrophobic drilling muds, and in aqueous systems, prevent clay from swelling. In asphalt emulsions as a cationic surfactant.

Several descriptions and illustrations have been presented to enhance understanding of the present invention. One skilled in the art will know that numerous changes and variations are possible without departing from the spirit of the invention. Each of these changes and variations are within the scope of the present invention.

I claim:

1) A disinfectant and its relevant salts of the following structure: 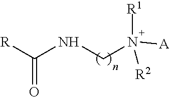
where R, R¹ and R² are independently chosen from alkyl, linear or branched, saturated or unsaturated, cyclic or acylic from 1 to 22 carbons, n is 2 or 3, and A is —(CH₂)₃Si(OH)₃, or —CH₂CH₂O⁻.

2) The disinfectant and its relevant salts of claim 1 where n=3, R¹ ═R²═—CH₃ and A=—(CH₂)₃Si(OH)₃.

3) The disinfectant and its relevant salts of claim 1, wherein R is such that the amide is formed from coconut fatty acid or oil, n=3, R¹═R²═—CH₃ and A=—(CH₂)₃Si(OH)₃.

4) The disinfectant and its relevant salts of claim 1 where R is —C₇H₁₅, n=3, R¹═R²═—CH₃ and A=—(CH₂)₃Si(OH)₃.

5) The disinfectant and its relevant salts of claim 1 where R is —C₄H₉, n=3, R¹═R²═—CH₃ and A=—(CH₂)₃Si(OH)₃.

6) The disinfectant and its relevant salts of claim 1 where n=3, R¹═R²═—CH₃ and A=—CH₂CH₂O⁻.

7) The disinfectant and its relevant salts of claim 1 where n=3, R¹═R²═—C₂H₅ and A=—CH₂CH₂O⁻.

8) The disinfectant and its relevant salts of claim 1 where n=3, R¹═R²═—C₂H₅ and A=—(CH₂)₃Si(OH)₃.

9) A disinfectant and its relevant salts of the following structure: 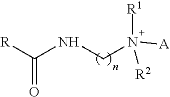
where R, R¹ and R² are independently chosen from alkyl, linear or branched, saturated or unsaturated, cyclic or acylic from 2 to 22 carbons, n is 2 or 3, and A is chosen from —CH₃, —CH₂CH₃, —CH₂C₆H₆, —CH(CH₂CH₃)C₆H₆, or —O⁻.

10) The disinfectant and its relevant salts of claim 9 where A=—CH(CH₂CH₃)C₆H₆.

11) The disinfectant and its relevant salts of claim 9 where A=—CH₂C₆H₆.

12) The disinfectant and its relevant salts of claim 9 where A=—CH₃.

13) The disinfectant and its relevant salts of claim 9 where A=—CH₂CH₃.

14) The disinfectant and its relevant salts of claim 9 where R¹═R²═—C₂H₅, A=—CH₂CH₃.

15) The disinfectant and its relevant salts of claim 9 where A=O⁻.
